(19) United States
(12) Patent Application Publication
(10) Pub. No.: US 2022/0222820 A1
(43) Pub. Date: Jul. 14, 2022

(54) IMAGE PROCESSING APPARATUS, IMAGE PROCESSING METHOD, AND PROGRAM

(71) Applicant: CANON KABUSHIKI KAISHA, Tokyo (JP)

(72) Inventor: Hiroyuki Omi, Kanagawa (JP)

(21) Appl. No.: 17/707,721

(22) Filed: Mar. 29, 2022

Related U.S. Application Data
(63) Continuation of application No. PCT/JP2020/035487, filed on Sep. 18, 2020.

(30) Foreign Application Priority Data
Sep. 30, 2019 (JP) 2019-180964

(51) Int. Cl. G06T 7/00; G16H 30/40

(52) U.S. Cl. CPC G06T 7/0012; G16H 30/40; G06T 2207/10116; G06T 2207/20004; G06T 2207/20084; G06T 2207/20081

(57) ABSTRACT

The present invention is directed to providing an image processing apparatus that improves efficiency of interpreting an abnormality candidate, also for a region not detected as an abnormality candidate by an abnormality candidate detection unit.

### CROSS-REFERENCE TO RELATED APPLICATIONS

This application is a Continuation of International Patent Application No. PCT/JP2020/035487, filed Sep. 18, 2020, which claims the benefit of Japanese Patent Application No. 2019-180964, filed Sep. 30, 2019, both of which are hereby incorporated by reference herein in their entirety.

### BACKGROUND OF THE INVENTION

### Field of the Invention

The present invention relates to an image processing apparatus, an image processing method, and a program which improve efficiency of interpreting an abnormality candidate, also for a region not detected as an abnormality candidate by an abnormality candidate detection unit.

### Background Art

In recent years, computer-aided detection/diagnosis (hereinafter, CAD) using machine learning has been utilized, and, in particular, CAD configured to perform supervised learning using a convolution neural network (hereinafter, CNN) has become rapidly widespread due to the high performance thereof (PTL 1). The CAD using the CNN determines, for example, an abnormality candidate for a captured image, using a learning parameter created by supervised learning. As a technique for a doctor to confirm the result of the determination by the CAD, a technique of emphasizing a detected region is also discussed (PTL 2).

### CITATION LIST

### Patent Literature

PTL 1: Japanese Patent Application Laid-Open No. 2017-45341

PTL 2: Japanese Patent Application Laid-Open No. 2018-192047

However, even if the result of the detection by the CAD can be partially emphasized, emphasis processing is not performed for a region not detected by the CAD, and therefore, interpretation efficiency can decrease.

### SUMMARY OF THE INVENTION

In view of the forgoing issue, the present invention is directed to providing an image processing apparatus that improves efficiency of interpreting an abnormality candidate, also for a region not detected as an abnormality candidate by a detector.

According to an aspect of the present invention, an image processing apparatus includes a medical image acquisition unit configured to acquire a medical image, an abnormality candidate detection unit configured to detect an abnormality candidate from the medical image acquired by the medical image acquisition unit, an image processing parameter setting unit configured to set an image processing parameter defining image processing to be applied to the medical image, based on the abnormality candidate detected by the abnormality candidate detection unit, an image processing unit configured to perform image processing on an image region that includes an image region corresponding to the abnormality candidate detected by the abnormality candidate detection unit, and is wider than the image region corresponding to the detected abnormality candidate, of the medical image, based on the image processing parameter set by the image processing parameter setting unit, and a display control unit configured to display the medical image subjected to the image processing by the image processing unit.

Further features of the present invention will become apparent from the following description of exemplary embodiments with reference to the attached drawings.

### BRIEF DESCRIPTION OF THE DRAWINGS

FIG. 1 is a diagram illustrating a configuration of an image processing apparatus in the present invention.

FIG. 2 illustrates an example of a hardware configuration of the image processing apparatus in the present invention.

FIG. 3 illustrates an image processing flow of the image processing apparatus in a first exemplary embodiment of the present invention.

FIG. 4A illustrates a training flow of an abnormality candidate detection unit in the image processing apparatus of the present invention.

FIG. 4B illustrates a training flow of the abnormality candidate detection unit in the image processing apparatus of the present invention.

FIG. 5 illustrates an image processing flow of the image processing apparatus according to a second exemplary embodiment in the present invention.

FIG. 6 illustrates a flow of image processing according to injury in the image processing apparatus of the present invention.

FIG. 7 is a diagram illustrating a display screen in the image processing apparatus of the present invention.

FIG. 8 is a diagram illustrating a display screen for selecting image processing in the image processing apparatus of the present invention.

### DESCRIPTION OF THE EMBODIMENTS

Exemplary embodiments according to the present invention will be described below with reference to the drawings. Exemplary embodiments are not limited to those described below, and any of those may be selectively implemented, or any plurality of exemplary embodiments among those may be combined and implemented.

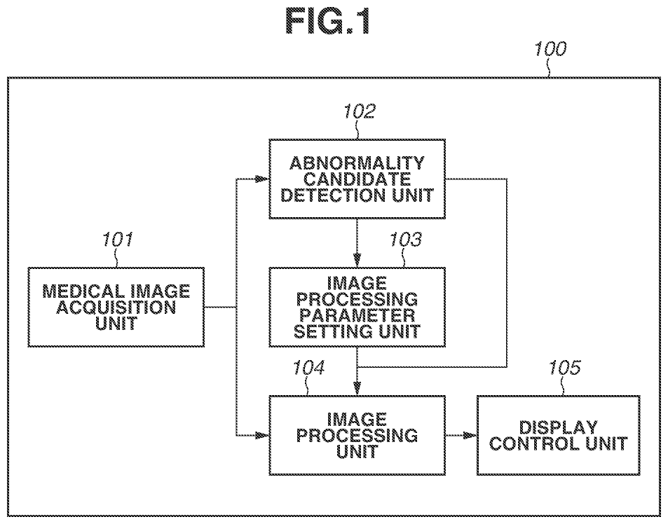

FIG. 1 is a diagram illustrating a configuration of an image processing apparatus 100 in the present invention. The image processing apparatus 100 has a medical image acquisition unit 101 that acquires an image, and an abnormality candidate detection unit 102 that detects an abnormality candidate. The image processing apparatus 100 further has an image processing parameter setting unit 103 that sets an image processing parameter based on the abnormality candidate detected by the abnormality candidate detection unit 102. The image processing apparatus 100 further has an image processing unit 104 that performs image processing based on the image processing parameter set by the image processing parameter setting unit 103. The image processing unit 104 executes the image processing on an image region that includes an image region corresponding to the abnormality candidate detected by the abnormality candidate detection unit 102, and is wider than the image region corresponding to the detected abnormality candidate, of a target image. The image processing apparatus 100 further has a display control unit 105 that displays a diagnosis image that is a medical image subjected to image processing.

The medical image acquisition unit 101 acquires a medical image such as an X-ray image from an external device, and outputs a preprocessed medical image. The abnormality candidate detection unit 102 takes the preprocessed medical image as input, and outputs an abnormality candidate detection result. The image processing parameter setting unit 103 takes the abnormality candidate detection result and an image processing preset parameter from the external device as input, and outputs an image processing update parameter. The image processing unit 104 takes the preprocessed medical image, the image processing update parameter, and the abnormality candidate detection result as input, performs the image processing on the medical image including an abnormality region detected by the abnormality candidate detection unit 102, and outputs the medical image subjected to the image processing. The display control unit 105 takes a diagnosis image that is the medical image subjected to the image processing, as input, and outputs an image processing result to a display device or the like.

In other words, the image processing apparatus 100 has the medical image acquisition unit 101 that acquires a medical image, and the abnormality candidate detection unit 102 that detects an abnormality candidate from the medical image acquired by the medical image acquisition unit 101. The image processing apparatus 100 further has the image processing parameter setting unit 103 that sets an image processing parameter defining image processing to be applied to the medical image, based on the abnormality candidate detected by the abnormality candidate detection unit 102. The image processing apparatus 100 further has the image processing unit 104 that performs image processing, based on the image processing parameter set by the image processing parameter setting unit 103. The image processing unit 104 performs the image processing on an image region that includes an image region corresponding to the abnormality candidate detected by the abnormality candidate detection unit 102, and is wider than the image region corresponding to the detected abnormality candidate, of the medical image. The image processing apparatus 100 further has the display control unit 105 that displays the medical image subjected to the image processing by the image processing unit 104.

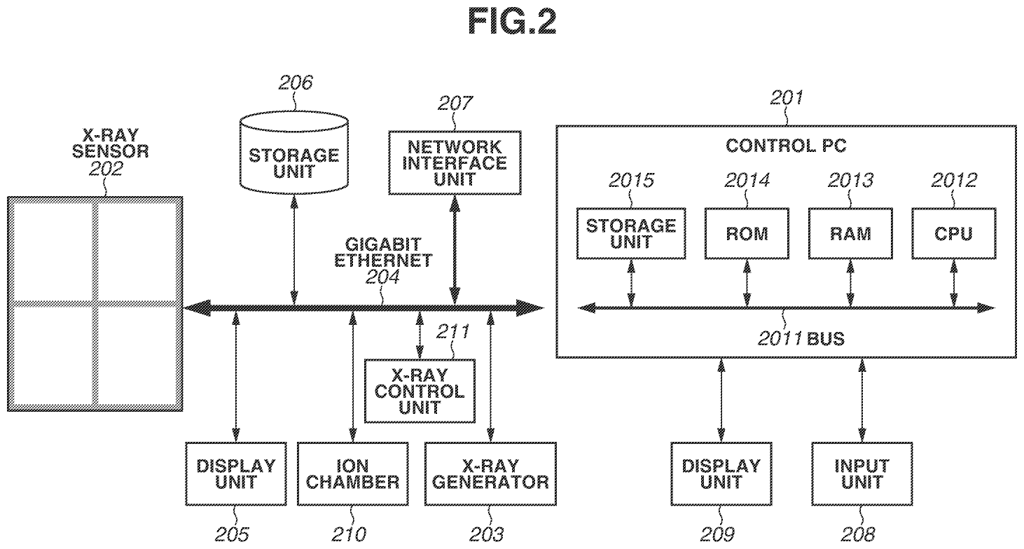

FIG. 2 illustrates an example of a configuration in a case where the configuration in FIG. 1 is implemented using a personal computer (PC). A control PC 201 and an X-ray sensor 202 are connected by a Gigabit Ethernet 204. In place of the Gigabit Ethernet 204, a Controller Area Network (CAN), an optical fiber, or the like may be used for a signal line. An X-ray generator 203, a display unit 205, a storage unit 206, a network interface unit 207, an ion chamber 210, and an X-ray control unit 211 are connected to the Gigabit Ethernet 204. The control PC 201 has, for example, a configuration in which a central processing unit (CPU) 2012, a random access memory (RAM) 2013, a read only memory (ROM) 2014, and a storage unit 2015 are connected to a bus 2011. Further, to the control PC 201, an input unit 208 is connected using Universal Serial Bus (USB) or Personal System/2 (PS/2), and a display unit 209 is connected using a Video Graphics Array (VGA) or Digital Visual Interface (DVI). Commands are sent to the X-ray sensor 202, the display unit 205, and the like via the control PC 201. In the control PC 201, processing content for each image capturing mode is stored in the storage unit 2015 as a software module, read by an instruction unit (not illustrated) into the RAM 2013, and executed. The medical image acquisition unit 101, the abnormality candidate detection unit 102, the image processing parameter setting unit 103, the image processing unit 104, and the display control unit 105 illustrated in FIG. 1 are stored in the storage unit 2015 as software modules. As a matter of course, in the present invention, the abnormality candidate detection unit 102 and the image processing unit 104 illustrated in FIG. 1 may each be implemented as an exclusive image processing board. The most appropriate implementation for the purpose may be achieved. The display control unit 105 illustrated in FIG. 1 controls display for the display unit 205 via the Gigabit Ethernet 204, or the display unit 209 connected to the control PC 201.

The operation of the image processing apparatus 100 illustrated in FIG. 1 and provided as a feature of the present exemplary embodiment, in an X-ray image processing apparatus having the above-described configuration, will be described in detail along with the following exemplary embodiments.

### First Exemplary Embodiment

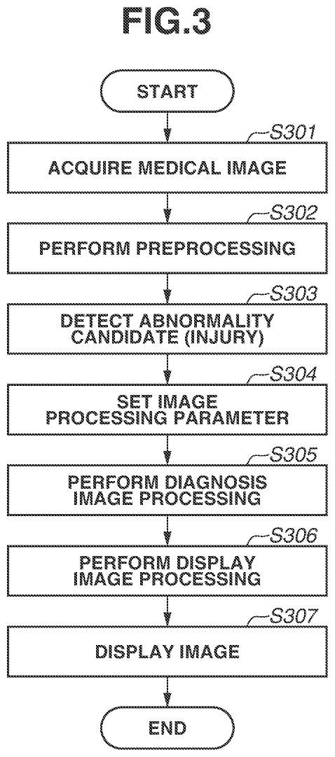

The description will be provided along a flow of processing of the present invention, with reference to the diagram illustrating the configuration of the image processing apparatus 100 in FIG. 1, and an overall flow of the image processing apparatus 100 in FIG. 3.

### (Step S301)

First, the medical image acquisition unit 101 acquires a medical image obtained using the X-ray sensor.

### (Step S302)

The medical image acquisition unit 101 performs preprocessing on the image acquired from the external device or the like, and outputs the preprocessed medical image. The preprocessing is, for example, processing for correction due to the characteristics of the sensor, which is processing of performing offset correction (dark current correction), gain correction, loss correction, and the like, thereby establishing a state in which a correlation with an adjacent pixel is maintained.

### (Step S303)

Next, the abnormality candidate detection unit 102 takes the medical image preprocessed by the medical image acquisition unit 101 as input, and detects an abnormality candidate (injury), thereby detecting an abnormality candidate (injury) region. For example, a convolution neural network (CNN) is used for the abnormality candidate (injury) detection unit 102, as one of machine learning techniques. When the CNN is used, training processing is performed beforehand and a parameter of the CNN is thereby determined. In the following, operation of the image processing apparatus 100 in training will be described, but in the present invention, a pre-trained model may be separately used, or a pre-trained model may be used as the abnormality candidate detection unit 102 that uses a model subjected to fine tuning or transfer learning. Further, the abnormality candidate detection unit 102 may be implemented not only by the CNN, but also by another type of deep learning technique, or a different machine learning technique such as support vector machines, Random Forest, or search tree, or by a combination of a plurality of machine learning techniques.

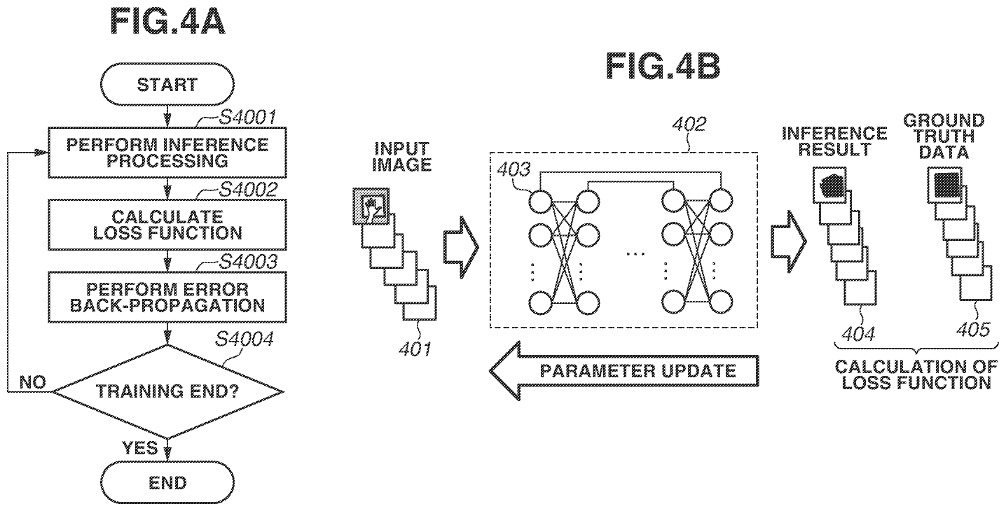

Here, operation when performing the training processing of the CNN is illustrated in FIG. 4A and FIG. 4B. The training processing is executed based on training data, in the case of supervised learning represented by the CNN. The training data is the pair of an input image 401 and ground truth data 405 corresponding thereto. Desirably, the ground truth data is, for example, a labeling image in which a desired region in an image is labeled with a given value, coordinate data in which a desired region is indicated by coordinates, an equation representing a straight line or curve indicating the boundary of a desired region, or the like. In the abnormality candidate (injury) detection processing, the ground truth data 405 may be, for example, a binary map image in which the abnormality candidate (injury) region in the input image 401 is 1 and the other regions are 0.

In the CNN in the abnormality candidate detection unit 102, inference processing is performed for the input image 401 in training, based on a parameter of a CNN 402, and an inference result 404 is output (step S4001). Here, the CNN 402 has a structure in which multiple processing units 403 are freely connected. For example, the processing unit 403 includes a convolution operation, normalization processing, or processing by an activation function such as ReLU or Sigmoid, and has a parameter group for describing each processing content. These can have any of various structures, for example, sets each performing processing in order of convolution processing→normalization→activation function are connected in about three to hundreds of layers.

Next, a loss function is calculated from the inference result 404 and the ground truth data 405 (step S4002). For the loss function, any function such as square error or cross entropy loss can be used. Error back-propagation starting from the loss function calculated in step S4002 is performed, and the parameter group of the CNN 402 is updated (step S4003).

Finally, whether to end the training is determined (step S4004), and the operation proceeds to step S401 in a case where the training is to be continued. The processing from step S4001 to step S4003 is repeated while the input image 401 and the ground truth data 405 are changed, so that parameter update of the CNN 402 is repeated to decrease the loss function, and the accuracy of the abnormality candidate detection unit 102 can be increased. In a case where the training has sufficiently progressed and it is determined to end the training, the processing ends. The end of the training is determined, for example, based on a criterion set according to a question, such as the inference result accuracy being a certain value or more without overtraining, or the loss function being a certain value or less.

### (Step S304)

Next, the image processing parameter setting unit 103 takes the injury candidate detection result and an image processing preset parameter from the external device as input, and outputs an image processing update parameter. The image processing preset parameter is a default parameter determined for each imaging site. The default parameter is an image processing parameter to express all diseases conceivable at each site. This is used in a medical examination or the like, and can comprehensively diagnose the whole. In a case where an abnormality (injury) is not detected as a result of the abnormality (injury) candidate detection, the image processing parameter setting unit 103 directly outputs the preset parameter as the image processing update parameter without changing the preset parameter. On the other hand, in a case where an abnormality candidate is detected, the image processing parameter setting unit 103 changes the preset parameter to a parameter dedicated to injury. For example, in a case where there is a bone fracture as the abnormality candidate, the parameter is changed to a bone fracture image processing parameter. The bone fracture image processing parameter is, for example, a parameter for intensifying edge enhancement and increasing the amount of compression of a dynamic range to make it easy to observe a bone part. For example, in a case where pneumothorax is present as the abnormality candidate, gradation processing is performed by, for example, bringing an average value of a region where pneumothorax is detected to the center of the gradation processing, to provide expression for making it easy to observe the inside of a lung field. In other words, the image processing parameter setting unit 103 changes the image processing parameter, in a case where the image processing parameter is set beforehand.

### (Step S305)

The image processing unit 104 takes the preprocessed medical image, the abnormality candidate detection result, and the image processing update parameter as input, performs the image processing, and creates a medical image subjected to the image processing (hereinafter, a diagnosis image). First, a diagnosis image is generated from the preprocessed medical image, using the image processing update parameter. The diagnosis image processing is processing for making it easy to view an abnormality candidate, and gradation processing, frequency processing, and the like are performed. In the image processing, the acquired image processing parameter is applied to a region wider than the region of the abnormality candidate detected by the abnormality candidate detection unit 102, so that the diagnosis image is generated. The image region to be the target of the image processing may be the entire preprocessed medical image output by the medical image acquisition unit 101, or, for example, the range of the image processing may be determined in consideration of the detected abnormality candidate, and the probability of the presence of an abnormality candidate. The image processing is executed on the image region that includes the detected abnormality candidate and is wider than the abnormality candidate, so that the image processing is executed also on a region that is actually an abnormality but not detected by the abnormality candidate detection unit 102, and as a result, the interpretation accuracy and the efficiency are expected to improve. In other words, the target of the image processing in the image processing unit 104 is the entire medical image in which the abnormality candidate is detected.

### (Step S306)

Here, the image processing update parameter is a parameter defining processing for making it easy to view an abnormality (injury), such as the gradation processing and the frequency processing. In other words, the image processing parameters such as the image processing update parameter and the image processing preset parameter are parameters defining at least one of the frequency processing and the gradation processing. Subsequently, display processing is further performed on the region determined to be the abnormality (injury) as the result of the abnormality (injury) candidate detection. For example, there is created an image in which the boundary of the abnormal (injury) region is displayed by a line having a specific color, or an image in which a mark such as “x” or an arrow is superimposed on the centroid portion of the injury region. A user can verify a region not detected as an abnormality region, in a comparative manner, based on the feature of the abnormality region.

### (Step S307)

Finally, the display control unit 105 outputs the diagnosis image to the display device. The display control unit 105 displays the diagnosis image by applying the image processing for easily confirming the region detected as the abnormality (injury) candidate, so that the abnormality candidate can be recognized. In addition, because the image processing for improving the readability of the detected abnormality candidate is executed also for the region where no abnormality candidate is detected, the region where the abnormality candidate is detected and the region where no abnormality candidate is detected can be compared. Therefore, the user less easily overlooks an oversight of the abnormality candidate detection unit 102, and, as a result, the efficiency of diagnosis by a doctor can be increased.

According to the present exemplary embodiment, the image processing parameter setting unit 103 sets the image processing parameter, based on the abnormality candidate detected by the abnormality candidate detection unit 102. Further, the image processing unit 104 executes the image processing on the image region including the abnormality candidate detected by the abnormality candidate detection unit 102. By this configuration, the image processing of emphasizing the abnormality candidate is performed also for the region not detected as the abnormality candidate by the abnormality candidate detection unit 102, and thus the interpretation efficiency improves.

### Second Exemplary Embodiment

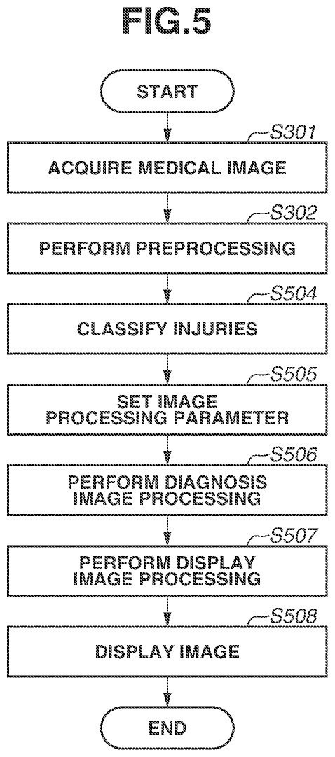

A different exemplary embodiment will be described with reference to the diagram illustrating the configuration in FIG. 1 and an overall flow in FIG. 5. In the present exemplary embodiment, the abnormality candidate detection unit 102 classifies a plurality of abnormality candidates based on a CNN. Step S301 and step S302 are similar to those in FIG. 3 in the first exemplary embodiment and thus will not be described.

### (Step S504)

In a case where classification into a plurality of injuries using a CNN is desirable, the abnormality candidate detection unit 102 determines the parameter of the CNN, for example, as follows. In a case where a CNN that performs binary classification of the presence/absence of each abnormality candidate is created, for example, a parameter for detecting only a bone fracture and using a bone fracture region as ground truth data is generated, and a parameter for detecting only pneumothorax and using a pneumothorax region as ground truth data is generated, so that the parameters are separately generated for the respective injuries.

Meanwhile, the plurality of abnormality candidates is classified by, for example, a single sorter, and thus the sorter learns features for classifying classification target abnormality candidates (classes) set in the sorter. Therefore, even in a case where the plurality of abnormality candidates has similar features (such as size, pixel value, position, and shape), it is easy to maintain the reliability of the classification. In a case where a sorter that classifies a plurality of classes is created, for example, a multiple-value map image in which ground truth data is a 1 for a bone fracture region, 2 for a pneumothorax region, 3 for a hemothorax region, and 0 for the other regions may be prepared, and parameters for detecting a plurality of injuries may be generated. In other words, the abnormality candidate detection unit 102 detects the abnormality candidates based on machine learning, and each of the abnormality candidates corresponds to a class of the machine learning.

The abnormality candidate detection unit 102 classifies the abnormality candidates based on the image acquired from the medical image acquisition unit 101, and outputs the detection result to the image processing parameter setting unit 103.

### (Step S505)

Next, the image processing parameter setting unit 103 takes the image processing preset parameter from the external device and the abnormality candidate detection result obtained by the abnormality candidate detection unit 102 as input, and outputs an image processing update parameter. The image processing update parameter is output for the number of the plurality of injuries into which the classification is performed by the abnormality candidate detection unit 102 in step S504.

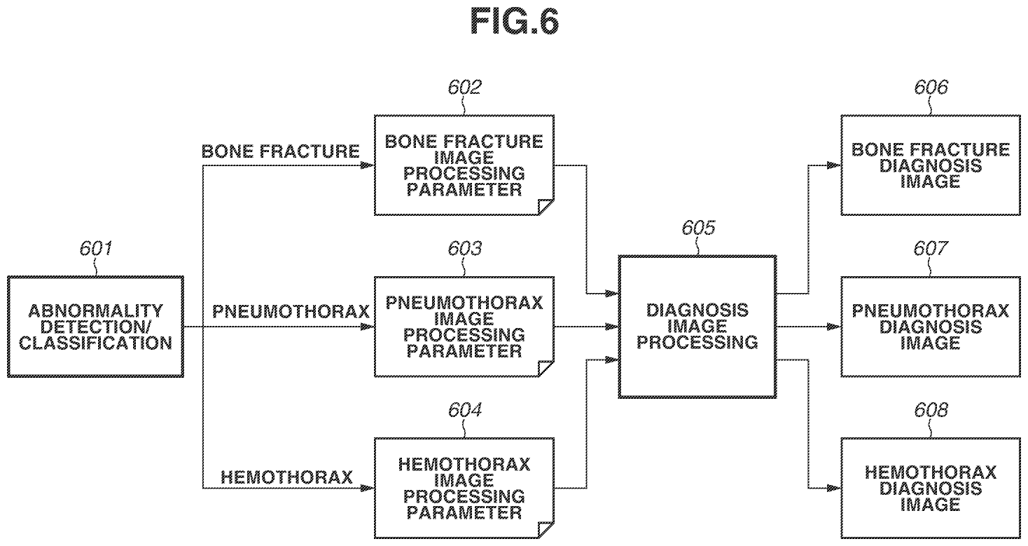

A case where a bone fracture, pneumothorax, and hemothorax are detected by the abnormality candidate detection unit 102 as a result of abnormality detection/classification 601 will be described with reference to FIG. 6. Here, a bone fracture image processing parameter 602, a pneumothorax image processing parameter 603, and a hemothorax image processing parameter 604 are generated and output. In a case where none of the abnormality candidates is detected as a result of the detection by the abnormality candidate detection unit 102, the image processing parameter may be set based on an instruction of a user from a graphical user interface (GUI) or the like, or the preset parameter may set. In other words, in a case where no abnormality candidate is detected by the abnormality candidate detection unit 102, the image processing apparatus 100 does not change the image processing parameter. In other words, in a case where a plurality of abnormality candidates is detected by the abnormality candidate detection unit 102, the image processing parameter setting unit 103 sets the image processing parameter for each of the plurality of abnormality candidates.

### (Step S506)

Next, the image processing unit 104 takes the preprocessed medical image, the abnormality candidate detection result, and the image processing update parameter as input, and creates an image for display. First, a diagnosis image is generated from the preprocessed medical image, using the image processing update parameter (step S506). In a case where the bone fracture, pneumothorax, and hemothorax are detected as the result of the abnormality detection/classification 601 as illustrated in FIG. 6, diagnosis image processing 605 generates a bone fracture diagnosis image 606 from the bone fracture image processing parameter 602. Further, a pneumothorax diagnosis image 607 is generated from the pneumothorax image processing parameter 603, and a hemothorax diagnosis image 608 is generated from the hemothorax image processing parameter 604. In other words, the image processing unit 104 outputs a plurality of image processing results, based on the image processing parameter set for each of the abnormality candidates.

### (Step S507)

Subsequently, display processing is performed on the region determined to be the abnormality (step S507). The bone fracture candidate detection result is superimposed on the bone fracture diagnosis image 606, the pneumothorax candidate detection result is superimposed on the pneumothorax diagnosis image 607, and the hemothorax candidate detection result is superimposed on the hemothorax diagnosis image 608.

The class configuration of the CNN, the training method, and the like are examples, and a plurality of CNNs may detect the respective abnormality candidates. Alternatively, the abnormality candidates detected by the plurality of CNNs may be compared for every pixel or every predetermined section, and the result thereof may be the result of the detection by the abnormality candidate detection unit 102.

### (Step S508)

Finally, the display control unit 105 outputs the plurality of diagnosis images sequentially to the display device (step S508). The plurality of diagnosis images may be switched by a switching operation on a user interface (UI), so that all the diagnosis images can be confirmed. The plurality of diagnosis images used for the image confirmation may be transferred to all picture achieving and communication systems (PACS). This makes it possible to perform confirmation while increasing diagnosis efficiency, even in a case where there is a plurality of lesions. In other words, a transfer unit that transfers the diagnosis image that is the medical image subjected to the image processing by the image processing unit 104 is included.

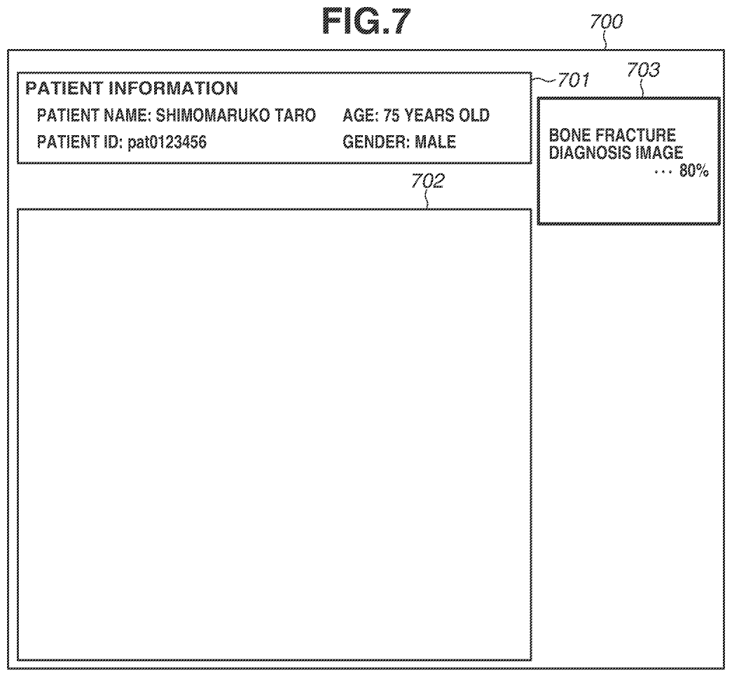

Here, an example of a display screen 700 displayed on the display device by the display control unit 105 according to the present invention will be described with reference to FIG. 7. The display screen 700 includes, a patient information portion 701 indicating the full name of a patient and the ID information of the patient, a diagnosis image display portion 702 generated by specific image processing, and an abnormality candidate information display portion 703 indicating the abnormality candidate corresponding to the diagnosis image display portion 702. For example, in a case where a bone fracture is detected by the abnormality candidate detection unit 102, the diagnosis image subjected to the bone fracture image processing by the image processing unit 104 is displayed in the diagnosis image display portion 702. Further, because the information corresponding to the bone fracture diagnosis image is displayed in the abnormality candidate information display portion 703, the user can recognize the abnormality candidate to be confirmed from the diagnosis image displayed in the diagnosis image display portion 702. In other words, the display control unit 105 displays the abnormality candidate detected by the abnormality candidate detection unit 102, and the medical image subjected to the image processing. Further, a certainty factor (to be described in detail below) for the classification of the sorter for the detected abnormality candidate may be displayed together therewith, in the abnormality candidate information display portion 703. Displaying the certainty factor for the classification together with the abnormality candidate makes it possible to confirm the diagnosis image obtained by subjecting the abnormality candidate to appropriate image processing, while considering the probability of being an abnormality candidate. In other words, the abnormality candidate detection unit 102 calculates the abnormality candidate, and the certainty factor of the classification for the abnormality candidate.

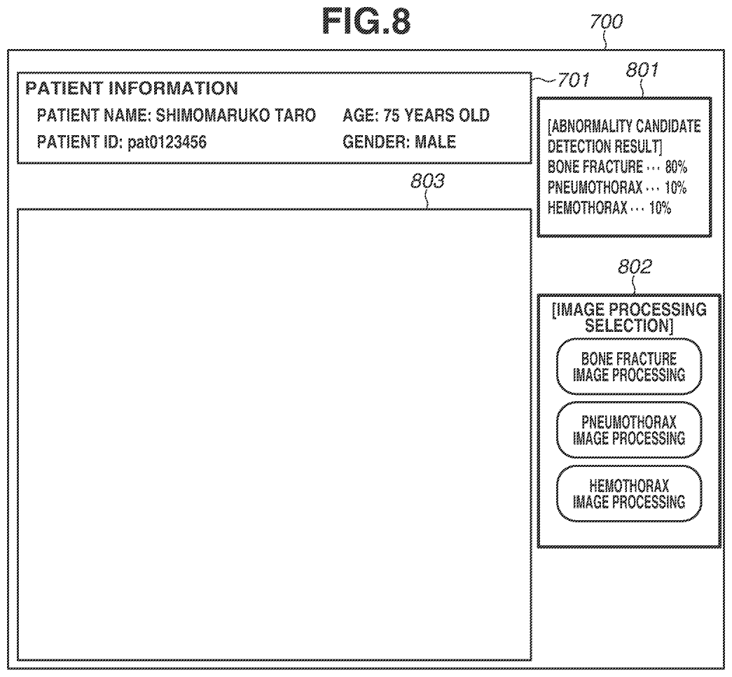

Further, a selection portion for enabling the user to select or switch the diagnosis image for display may be included. Referring to FIG. 8, the display screen 700 displayed by the display control unit 105 further displays an abnormality candidate detection result display portion 801 for displaying the degree of certainty of the sorter for the abnormality candidate detection. In addition, a selection portion 802 for selecting image processing to be performed on a target medical image is displayed, and further, a diagnosis image display portion 803 for displaying the diagnosis image that is the result of the selected image processing is displayed. In a case where the result of the detection by the abnormality candidate detection unit 102 is not sufficiently reliable, or a case where the user wants to perform desired image processing, desired image processing can be selected in the selection portion 802, so that reduction of diagnosis errors and improvement of diagnosis efficiency are expected. In other words, the display control unit 105 has the selection portion 802 for selecting an image processing result to be displayed, in a case where the output by the image processing unit 104 is a plurality of image processing results. If the plurality of diagnosis images can be identifiably displayed, the images may be displayed in the same screen or at the same time, i.e., switching the diagnosis image in the diagnosis image display portion 803 displayed by the selection portion 802 to another may not be performed.

In other words, the display control unit 105 displays each of the plurality of image processing results (the diagnosis image display portion 803) identifiably.

### Third Exemplary Embodiment

As described in the second exemplary embodiment, in the case of having the plurality of abnormality candidates as the classes of the sorter, the abnormality candidate detection unit 102 detects the plurality of abnormality candidates based on the CNN.

In the present exemplary embodiment, output to the CNN of the abnormality candidate detection unit 102, and an example of display control performed by the display control unit 105 in a case where a plurality of abnormality candidates is detected will be described.

In a case where an object of input to the sorter is an image, the CNN can set the unit of output of a classification result to a pixel unit, image region unit, image unit, or the like. The CNN in the abnormality candidate detection unit 102 in this form is applicable to any of these. In addition, the CNN sets a certainty factor for each object of the classification result. The certainty factor represents the certainty of being an abnormality candidate (injury). The certainty factor in the CNN is, for example, a softmax value calculated by a softmax function. The detection of the region corresponding to the abnormality candidate is not limited to the output result of the last layer of the CNN, and, for example, a value of an output result of a middle layer may be used. Further, processing for detection of a plurality of abnormality candidates may be performed, and a score obtained by weighting the detection result may be used.

Based on the abnormality candidates detected by the abnormality candidate detection unit 102, the image processing parameter setting unit 103 sets an image processing parameter corresponding to each of the abnormality candidates. Subsequently, the image processing unit 104 executes image processing, so that a plurality of diagnosis images that is a plurality of medical images subjected to the image processing is generated.

Here, the display control unit 105 acquires the certainty factor output by the abnormality candidate detection unit 102, and outputs an image corresponding to the abnormality candidate of the highest certainty factor. In other words, the display control unit 105 displays the diagnosis image as the image processing result corresponding to the abnormality candidate of the highest certainty factor, on the display device.

Alternatively, the display control unit 105 may comparably display a plurality of images each corresponding to the abnormality candidate having the certainty factor more than or equal to a threshold, or may display the plurality of images sequentially.

In the case of displaying the plurality of image sequentially, the display control unit 105 displays the plurality of images in, for example, descending order of certainty factor output by the abnormality candidate detection unit 102. In other words, in a case where the output by the image processing unit 104 is the plurality of medical images (diagnosis images) subjected to the image processing, the display control unit 105 determines the display order of the plurality of medical images (diagnosis images) according to the certainty factor. Alternatively, the degree of urgency or the degree of progress of each of the abnormality candidates may be output and the display order may be determined based on this output. The display order may be determined based on each of the certainty factor, the degree of urgency, and the degree of the progress alone, or the display order may be determined based on an evaluation value obtained by a combination of these. In a case where a plurality of images is present, the display control unit 105 displays the images based on the certainty factor, the degree of urgency, and/or the degree of the progress, so that a user can first confirm the abnormality candidate (injury) given the highest priority for treatment. The degree of urgency or the degree of progress is, for example, the result of applying labels of ground truth data, such as bone fracture—level 1, bone fracture—level 2, and bone fracture—level 3, to each level of the degree of urgency or the degree of progress, performing training, and performing classification by the trained sorter, in the sorter that classifies bone fracture, pneumothorax, and hemothorax into classes. Alternatively, the degree of urgency or the degree of progress may be obtained by additionally providing a different sorter (CNN) that takes the region of the abnormality candidate detected by the sorter (CNN) in the abnormality candidate detection unit 102 as input, and outputs the degree of urgency or the degree of progress.

Moreover, a value obtained further by multiplying the certainty factor for the classification result of the sorter by the reliability of the sorter itself may be used as the output by the abnormality candidate detection unit 102. For example, the classification accuracy of the sorter can be insufficient for a specific abnormality candidate, even in a case where the certainty factor for the classification result of the sorter is very high. In such a case where the classification accuracy is not sufficient, for example, the certainty factor of the sorter is multiplied by the reliability of the sorter itself. Taking the reliability of the sorter into consideration makes it possible to consider classification results by a plurality of sorters, or noise or bias to the result of classification into a plurality of classes, so that images can be displayed in appropriate order for a user. As a matter of course, the order of display by the display control unit 105 is not limited to this form. For example, display may be performed according to a display order set beforehand by the user or the like.

### Fourth Exemplary Embodiment

Another exemplary embodiment different in terms of the image display in step S307 of the first exemplary embodiment will be described with reference to the diagram illustrating the configuration in FIG. 1 and the overall flow in FIG. 3.

Step S301 to step S303 are similar to those of the first exemplary embodiment and thus will not be described.

The image processing parameter setting unit 103 takes the image processing preset parameter from the external device and the abnormality candidate detection result as input, and outputs the image processing preset parameter and the image processing update parameter (S304). In a case where there is no injury, only the image processing preset parameter is output. In a case where there is an abnormality candidate, a parameter dedicated to this abnormality candidate is output as the image processing update parameter, together with the image processing preset parameter.

Next, the image processing unit 104 takes the preprocessed medical image, the abnormality candidate detection result, the image processing update parameter, and the image processing preset parameter as input, and creates a diagnosis image. First, the image processing unit 104 generates a diagnosis image from the preprocessed medical image, using the image processing update parameter (step S305). Further, the image processing unit 104 creates a diagnosis image, using the image processing preset parameter. Subsequently, for the diagnosis image using the update parameter, the region determined to be the abnormality candidate is subjected to the display processing (step S306).

Finally, the display control unit 105 outputs the plurality of display images sequentially to the display device (step S307). The diagnosis images using the update parameter are sequentially displayed. Subsequently, the diagnosis image using the preset parameter is displayed as the last image to confirm the whole at the end. In a case where an instruction to display the diagnosis image using the preset parameter is provided from a UI, the image is displayed accordingly. The effect of preventing oversight can be obtained by performing the last confirmation using the image processing preset parameter for enabling the confirmation of the whole. In other words, the display control unit 105 displays the image processing result (diagnosis image) subjected to the image processing using the image processing parameter before the image processing parameter is changed.

### Modification

If the number of abnormality candidates detected by the abnormality candidate detection unit 102 is large, it is conceivable that the number of diagnosis images to be generated to correspond to the respective abnormality candidates also increases. In a case where the number of diagnosis images is a burden on the user, the image processing parameter setting unit 103 may compare the differences between the image processing parameters corresponding to the plurality of abnormality candidates. Subsequently, predetermined image processing may be performed for each group of abnormality candidates corresponding to similar image processing parameters, and the display control unit 105 may perform display based on the result thereof. The user may set a group of abnormality candidates corresponding to similar image processing parameters.

### Other Exemplary Embodiments

The present invention is also implemented by executing the following processing. Specifically, software (a program) that implements the function of each of the exemplary embodiments described above is supplied to a system or apparatus via a network or any of various storage media, and a computer (or a CPU or a micro processing unit (MPU)) of the system or apparatus reads out the program and executes the program.

The present invention is not limited to the above embodiments and various changes and modifications can be made within the spirit and scope of the present invention. Therefore, to apprise the public of the scope of the present invention, the following claims are made.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, the efficiency of interpreting an abnormality candidate can be improved also for a region not detected by a detector, by performing image processing on a medical image based on an abnormality candidate detected by an abnormality candidate detection unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus comprising:
a medical image acquisition unit configured to acquire a medical image;
an abnormality candidate detection unit configured to detect an abnormality candidate from the medical image acquired by the medical image acquisition unit;
an image processing parameter setting unit configured to set an image processing parameter defining image processing to be applied to the medical image, based on the abnormality candidate detected by the abnormality candidate detection unit;
an image processing unit configured to perform image processing on an image region that includes an image region corresponding to the abnormality candidate detected by the abnormality candidate detection unit, and is wider than the image region corresponding to the detected abnormality candidate, of the medical image, based on the image processing parameter set by the image processing parameter setting unit; and
a display control unit configured to display the medical image subjected to the image processing by the image processing unit.

2. The image processing apparatus according to claim 1, wherein a target of the image processing in the image processing unit is the entire medical image.

3. The image processing apparatus according to claim 1, wherein the image processing parameter is a parameter defining at least one of frequency processing and gradation processing.

4. The image processing apparatus according to claim 1, wherein the image processing parameter setting unit changes the image processing parameter, in a case where the image processing parameter is set beforehand.

5. The image processing apparatus according to claim 1, wherein, in a case where a plurality of abnormality candidates is detected by the abnormality candidate detection unit, the image processing parameter setting unit sets an image processing parameter for each of the plurality of abnormality candidates.

6. The image processing apparatus according to claim 1, wherein the display control unit displays the abnormality candidate detected by the abnormality candidate detection unit and the medical image subjected to the image processing.

7. The image processing apparatus according to claim 5, wherein the image processing unit performs image processing, based on the image processing parameter set for each of the plurality of abnormality candidates, and outputs a plurality of medical images subjected to the image processing.

8. The image processing apparatus according to claim 7, wherein the display control unit identifiably displays each of the plurality of medical images subjected to the image processing.

9. The image processing apparatus according to claim 1, wherein the abnormality candidate detection unit calculates the abnormality candidate, and a certainty factor of classification for the abnormality candidate.

10. The image processing apparatus according to claim 9, wherein the display control unit displays the medical image subjected to the image processing and corresponding to the abnormality candidate of the highest certainty factor.

11. The image processing apparatus according to claim 9, wherein, in a case where output by the image processing unit is a plurality of medical images subjected to the image processing, the display control unit determines a display order of the plurality of medical images subjected to the image processing, based on the certainty factor.

12. The image processing apparatus according to claim 4, wherein the display control unit displays the medical image subjected to the image processing using the image processing parameter before the image processing parameter is changed, on a display device.

13. The image processing apparatus according to claim 1, wherein the abnormality candidate detection unit detects abnormality candidates based on machine learning, and each of the abnormality candidates corresponds a class of the machine learning.

14. The image processing apparatus according to claim 1, wherein the display control unit has a selection portion for selecting a medical image subjected to the image processing and to be displayed, in a case where output by the image processing unit is a plurality of medical images subjected to the image processing.

15. The image processing apparatus according to claim 1, further comprising a transfer unit configured to transfer the medical image subjected to the image processing by the image processing unit.

16. An image processing method comprising:
acquiring a medical image;
detecting an abnormality candidate from the acquired medical image;
setting an image processing parameter defining image processing to be applied to the medical image, based on the detected abnormality candidate;
performing image processing on an image region that includes an image region corresponding to the detected abnormality candidate, and is wider than the image region corresponding to the detected abnormality candidate, of the medical image, based on the set image processing parameter; and
displaying the medical image subjected to the image processing.

17. The image processing method according to claim 16, wherein a target of the image processing is the entire medical image.

18. A program that causes a computer to execute the image processing method according to claim 16.
